(19) United States
(12) Patent Application Publication
(10) Pub. No.: US 2026/0248832 A1
(43) Pub. Date: Aug. 27, 2026

(54) COMPOSITION COMPRISING CYTIDINE ANALOGS AND USES AND METHODS THEREOF

(71) Applicant: UNIVERSITÄT HEIDELBERG, Heidelberg (DE)

(72) Inventors: Elena-Sophie PRIGGE, Heidelberg (DE); Magnus VON KNEBEL DÖBERITZ, Heidelberg (DE); Lukas UEBBING, Mainz (DE); Peter LANGGUTH, Biebergemünd (DE)

(21) Appl. No.: 18/853,853

(22) PCT Filed: Apr. 24, 2023
(86) PCT No.: PCT/EP2023/060649

§ 371 Date: Oct. 3, 2024

(30) Foreign Application Priority Data
Apr. 22, 2022 (EP) 22169454.0

(51) Int. Cl. A61K 31/706; A61J 1/05; A61K 9/00; A61K 9/06; A61K 47/10; A61K 47/20; A61P 35/00

(52) U.S. Cl. CPC A61K 31/706; A61J 1/05; A61K 9/0014; A61K 9/06; A61K 47/10; A61K 47/20; A61P 35/00

(57) ABSTRACT

The present invention provides a composition comprising a cytidine analog, in particular decitabine or azacitidine. In particular, the present invention provides a composition comprising a cytidine analog, in particular decitabine or azacitidine, which is useful for topical application. The present invention also provides the use of such compositions for medical conditions in the keratinizing and non-keratinizing epithelium/skin, such as the treatment of human papillomavirus (HPV)-related pre-cancerous conditions.

The present invention relates to the field of compositions comprising cytidine analogs, in particular decitabine or azacitidine. In particular, the present invention relates to a composition comprising a cytidine analog, in particular decitabine or azacitidine, which is useful for topical application and a package containing the same. The present invention also relates to the use of such compositions for the treatment of human papillomavirus (HPV)-related pre-cancerous conditions.

Oncogenic types of human papillomaviruses (HPV) can cause a variety of pre-cancerous conditions in the anogenital region and invasive tumors in the anogenital and head and neck regions. The development and maintenance of the malignant phenotype in HPV-related tumors is essentially dependent on the continued expression of HPV E6 and E7 oncoproteins (von Knebel Doeberitz M, Oltersdorf T, Schwarz E, Gissmann L. Correlation of modified human papilloma virus early gene expression with altered growth properties in C4-1 cervical carcinoma cells. Cancer Res. 1988 Jul. 1; 48 (13): 3780-6; reviewed in zur Hausen H. Papillomaviruses in anogenital cancer as a model to understand the role of viruses in human cancers. Cancer Res. 1989 Sep. 1; 49 (17): 4677-81). Previous research has shown that certain epigenetic modifications of the HPV genome itself-specifically hypermethylation within the viral upstream regulatory region (URR)—can trigger uncontrolled expression of HPV E6/E7 oncoproteins (Vinokurova S, von Knebel Doeberitz M. Differential methylation of the HPV 16 upstream regulatory region during epithelial differentiation and neoplastic transformation. PLoS One. 2011; 6 (9): e24451. doi: 10.1371/journal.pone.0024451). Based thereon, it has been hypothesized that treatment of HPV-transformed cells with demethylating agents, such as decitabine (5-aza-2′-deoxycytidine, DAC), may lead to regression of the malignant phenotype by removal of aberrant methylation patterns and subsequent downregulation of HPV E6/E7 oncoprotein expression in affected cells. This hypothesis was confirmed in vitro using a variety of HPV-transformed cell lines (Stich M, Ganss L, Puschhof J, Prigge E S, Reuschenbach M, Guiterrez A, Vinokurova S, von Knebel Doeberitz M. 5-aza-2′-deoxycytidine (DAC) treatment downregulates the HPV E6 and E7 oncogene expression and blocks neoplastic growth of HPV-associated cancer cells. Oncotarget. 2016 Jul. 16; 8 (32): 52104-52117).

In the prior art, decitabine-containing drugs are usually developed for injection or infusion, in particular for intravenous administration. For example, US 2003/229047 A1 describes a liquid formulation, which comprises decitabine solvated in a solvent comprising glycerin, propylene glycol, polyethylene glycol, or combinations thereof. However, the compositions of US 2003/229047 A1 are not only liquid, but they also appear to be unstable. Likewise, the formulation disclosed in WO 2006/071491 A1 does apparently not fulfill the stability requirements for decitabine. In addition, it contains at least 60% water and is, thus, also liquid. While WO 2014/064717 A1 focuses on the stability problem, the decitabine compositions disclosed therein are also formulated for intravenous administration and are, thus, liquid formulations. Likewise, the lipid nanoparticles described as carriers in WO 2012/073125 are developed for intravenous administration specifically for the treatment of thyroid cancer.

However, systemic administration of drugs containing demethylating agents, such as decitabine or azacitidine, harbors the risk of causing systemic side effects since drug delivery and uptake are not confined to a distinct target location.

In view of the above, it is the object of the present invention to overcome the drawbacks of prior art and to provide a composition comprising a cytidine analog, in particular decitabine or azacitidine, which is useful for topical application and/or efficacy. Local (topical) application is advantageous to achieve a local/topical effect, e.g. on transformed cells of HPV-related pre-cancerous conditions, while minimizing the risk for systemic drug uptake and associated side effects. However, the above-described prior art formulations of decitabine were developed primarily for injection or infusion, in particular for intravenous administration. Therefore, the prior art formulations of decitabine are usually liquid compositions, which is advantageous for intravenous administration, but not for local/topical administration.

In addition, it is well-known that dissolved decitabine exhibits marked instability under a variety of conditions including oxidation or hydrolytic degradation in the alkaline and acidic pH range (Rogstad D K, Herring J L, Theruvathu J A, Burdzy A, Perry C C, Neidigh J W, Sowers L C. Chemical decomposition of 5-aza-2′-deoxycytidine (Decitabine): kinetic analyses and identification of products by NMR, HPLC, and mass spectrometry. Chem Res Toxicol. 2009 June; 22 (6): 1194-204). Furthermore, HPV-related pre-cancerous conditions develop primarily in the keratinizing or non-keratinizing epithelium of the anogenital region and are often associated with local irritation, such as itching and pain. In view thereof, it is also an object of the present invention to provide a composition comprising a cytidine analog, in particular decitabine or azacitidine, in which the cytidine analog, in particular decitabine or azacitidine, is stably formulated. Furthermore, it is an object of the present invention to provide a composition comprising a cytidine analog, in particular decitabine or azacitidine, which is formulated such that the cytidine analog, in particular decitabine or azacitidine, can penetrate into the lower layers of the epithelium after local application. In addition, it is an object of the present invention to provide a composition comprising a cytidine analog, in particular decitabine or azacitidine, as well as a pharmaceutically acceptable carrier, vehicle or diluent, which enables a stable preparation of the active agent, namely, the cytidine analog, in particular decitabine or azacitidine, over a broad range of final concentrations. It is also an object of the present invention to provide a composition comprising a cytidine analog, in particular decitabine or azacitidine, which exhibits a favorable consistency for local/topical application, in particular a consistency that allows the product to be spread well for comfortable and non-irritating application to an epithelial lesion.

These objects are achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

### Compositions

In a first aspect the present invention provides a pharmaceutical composition comprising:
decitabine or azacitidine, or a pharmaceutically acceptable salt thereof;
polyethylene glycol (PEG); and
dimethyl sulfoxide (DMSO).

The pharmaceutical composition is suitable for topical application. To this end, the pharmaceutical composition is typically semi-solid at room temperature, e.g. at about 25° C. Thereby, the composition can be easily and evenly applied topically to a specific target region, e.g. to a lesion, and it remains essentially at the area, to which it has been applied (instead of running down). Moreover, for topical application, the composition is preferably well deformable and/or easily spreadable at room temperature, e.g. at about 25° C. Thereby, it allows for easy and comfortable application and easy removal from the tube/primary packaging, while irritation or pain upon application is avoided. Accordingly, the composition is preferably not a liquid composition (as compositions developed, e.g., for injection or intravenous administration). Instead, the composition of the invention for topical application has a higher viscosity as compared to liquid compositions (e.g., for injection), which is to ensure specific targeting of affected areas, easy application (e.g. with a finger) and even spreading during topical administration. Preferably, the composition is semi-solid at room temperature.

Accordingly, the viscosity of the composition should on the one hand be sufficiently high to substantially prevent running or unintentional spreading of the composition from the area where it is applied to other areas. On the other hand, the viscosity of the composition should be sufficiently low to enable the patient to readily remove the required dose of the composition from the container in which it is available (e.g. a tube or the like), to apply it evenly over the affected area to ensure an even dosing of the active component, and to avoid or minimize friction at the site of application which could cause local irritation and/or pain. To obtain a viscosity suitable for topical application (in particular, a semi-solid state of the composition), a thixotropic agent is included as viscosity-increasing agent, such that the composition is typically in the form of an ointment. A thixotropic agent has the advantage of being readily applied, while on standing, such as after application, the viscosity increases so that the composition will typically not run or unintentionally spread from the affected areas of the skin on which it is applied to unaffected areas. In the composition of the present invention, polyethylene glycol (PEG) is used as thixotropic agent. It is understood that for the pharmaceutical composition, the PEG (as well as any other component) included in the composition is pharmaceutical-grade compound, i.e. having a chemical purity acceptable for pharmaceutical applications.

In the composition of the invention, the active pharmaceutical ingredient is typically decitabine or azacitidine (or the salt thereof). In some embodiments, the composition comprises no further active ingredients, i.e. other than decitabine or azacitidine (or the salt thereof). In other embodiments, the composition may comprise an additional active ingredient, i.e. in addition to decitabine or azacitidine (or the salt thereof). However, PEG, DMSO and other excipients, solvents, diluents or vehicles are usually not considered as active pharmaceutical ingredients.

Decitabine and azacitidine are chemical analogs of the nucleoside cytidine (cytidine analogs). Decitabine is the deoxy derivative of azacitidine.

Decitabine, also referred to as 5-aza-2′-deoxycytidine, is shown in formula (1) below:

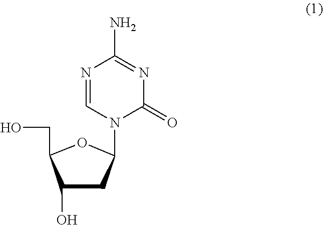

Azacytidine, also referred to as 5-Azacytidine, Ladakamycin, or 4-Amino-1-β-D-ribofuranosyl-s-triazin-2 (1H)-one, is shown in formula (2) below:

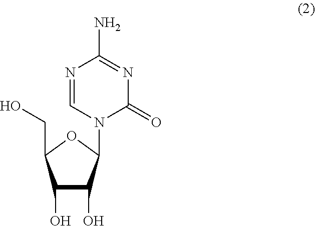

Decitabine or azacytidine may be in the form of a (preferably pharmaceutically acceptable) salt. Examples thereof include, but are not limited to pharmaceutically acceptable salts prepared from an inorganic acid or an organic acid. Examples of such inorganic acids include, but are not limited to, hydrochloric and hydrobromic acid. Examples of such organic acids include, but are not limited to, aliphatic, cycloaliphatic, aromatic, arylaliphatic, heterocyclic, carboxylic and sulfonic organic acids, such as formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, [beta]-hydroxybutyric, malonic, galactic, and galacturonic acid. In some embodiments, the acid is selected from the group consisting of hydrochloric acid, L-lactic acid, acetic acid, phosphoric acid, (+)-L-tartaric acid, citric acid, propionic acid, butyric acid, hexanoic acid, L-aspartic acid, L-glutamic acid, succinic acid, EDTA, maleic acid, methanesulfonic acid, hydrochloric acid, hydrobromic acid, nitric acid, nitrous acid, sulfuric acid, sulfurous acid, phosphorous acid, perchloric acid, chloric acid, chlorous acid, carboxylic acid, sulfonic acid, ascorbic acid, carbonic acid and fumaric acid. The sulfonic acid may be selected from the group consisting of ethanesulfonic, 2-hydroxyethanesulfonic, and toluenesulfonic acid.

Preferably, the pharmaceutical composition comprises decitabine or azacitidine (or the salt thereof) at a concentration of 0.005-100 mM, more preferably at a concentration of 0.01-50 mM, even more preferably at a concentration of 0.05-25 mM, and still more preferably at a concentration of 0.1-10 mM (0.0228 mg/g-2.28 mg/g). As shown in the appended examples, topical application of the composition of the invention containing decitabine or azacitidine at such doses results in a dose-dependent tumor cell degradation in a model of HPV-transformed pre-cancerous tumors.

In a further aspect, the present invention also provides a pharmaceutical composition comprising 0.1-10 mM (0.0228 mg/g-2.28 mg/g) decitabine or azacitidine or a pharmaceutically acceptable salt thereof for medical use, wherein the pharmaceutical composition is applied topically.

Such a pharmaceutical composition for use as described herein preferably comprises polyethylene glycol (PEG) and dimethyl sulfoxide (DMSO). Accordingly, the description of the pharmaceutical composition as provided herein likewise applies to the pharmaceutical composition for use and vice versa.

As described above, for topical application the viscosity of the composition should on the one hand be sufficiently high to substantially prevent running or unintentional spreading of the composition from the area where it is applied to other areas and, on the other hand, the viscosity of the composition should be sufficiently low to enable the patient to readily remove the required dose of the composition from the container in which it is available (e.g. a tube or the like), to apply it evenly over the affected area to ensure an even dosing of the active component, and to avoid or minimize friction at the site of application, such as the anogenital area, which could cause local irritation and/or pain. The viscosity of the product is characterized by its complex viscosity η* that combines G′ (elastic modulus) and G″ (viscous modulus) of said composition. Therefore, the compositions of the invention as described herein have preferably a complex viscosity from about 1,000 Pa·s to about 25,000 Pa·s at 25° C., preferably from about 2,000 Pa·s to about 15,000 Pa·s at 25° C., and even more preferably from about 3,000 Pa·s to about 11,000 Pa·s at 25° C. The complex viscosity is usually determined under oscillations. In particular, the complex viscosity may suitably be determined at room temperature (about 25° C.) under an oscillating ramp at a predefined strain amplitude fixed at 5 Pa and at a fixed frequency set at 1 Hz using a Rheometer (e.g., Anton Paar), in particular involving a cone-plate geometry with a gap of 400 nm. The complex viscosity value may be the mean of 10 values collected for 60 seconds. A specific example for determining the complex viscosity is provided in Example 2, method C.

To obtain the envisaged complex viscosity, PEG is used in the compositions of the invention as thixotropic (thickening) agent. PEGs are typically prepared by polymerization of ethylene oxide and are commercially available over a wide range of molecular weights from 100 g/mol to 10,000,000 g/mol. The numbers, which are usually included in the names of PEGs indicate their average molecular weights (e.g., a PEG with n=9 would have an average molecular weight of approximately 400 g/mol, and would be labeled PEG 400; PEG 300 has a molecular weight of approx. 300 g/mol, PEG 1000 has a molecular weight of approx. 1000 g/mol, PEG 1500 has a molecular weight of approx. 1500 g/mol and the like). PEGs of different molecular weights are referred to herein as different PEG components. The PEG component of the composition of the invention allows to provide the semi-solid ointment base of the composition for adjusting the viscosity within the desired range.

The amount of PEG in the composition of the invention may be at least 30% (by weight of the finished product), at least 35% (by weight of the finished product), at least 40% (by weight of the finished product), at least 45% (by weight of the finished product), at least 50% (by weight of the finished product), at least 55% (by weight of the finished product), at least 60% (by weight of the finished product), at least 65% (by weight of the finished product), or even higher. Preferably, the amount of PEG in the composition of the invention is at least 70% (by weight of the finished product), preferably at least 75% (by weight of the finished product), more preferably at least 80% (by weight of the finished product), even more preferably at least 85% (by weight of the finished product), still more preferably at least 90% (by weight of the finished product), particularly preferably at least 91% or 92% (by weight of the finished product), and most preferably at least 93% or 94% (by weight of the finished product). In some embodiments, the amount of PEG in the composition of the invention may be from 94% to 95% (by weight of the finished product).

In some embodiments, a single PEG component may be used, in particular to provide the composition in a semi-solid form. To this end, PEG 800-PEG 1500 may be used as single-component PEG, for example PEG 800, PEG 900, PEG 1000, PEG 1100, PEG 1200, PEG 1300, PEG 1400, PEG 1500. In some embodiments, the single PEG component may be combined with a higher amount of DMSO to provide a composition, which is more fluid as compared to the single PEG component alone. Thereby, the higher the molecular weight of the PEG (i.e., the more “solid” the PEG), the higher amount of DMSO may be used. For example, if PEG 1500 is used as single PEG component, it may be used at 40-45% (by weight of the finished product), while DMSO may be used in such a composition at 53-58% (by weight of the finished product). For other PEGs with lower molecular weight (used as single PEG component), the amount of DMSO may be reduced (in comparison to the PEG 1500 example above).

Preferably, the compositions of the invention comprise at least two distinct PEG components. For example, the compositions of the invention may comprise 2, 3, 4, 5, 6, 7, 8, 9 or more (different) PEG components. Preferably, the compositions of the invention comprise (exactly) two or three distinct PEG components, even more preferably, the compositions of the invention comprise (exactly) two distinct PEG components.

The first PEG component has preferably a solid physical state at 25° C. and the second PEG component comprised in the composition has preferably a liquid physical state at 25° C., i.e. a solid PEG is preferably combined with a liquid PEG.

Without being bound to any theory, the present inventors assume that in such a combination the shorter chains of the liquid PEG component, e.g. PEG 200-600, are arranged in the parallel crystal layers (or folded layers) of the solid PEG component, e.g. PEG 1000-8000, in particular of the terminal OH-groups, thereby resulting in swelling. Without being bound to any theory, it is assumed that such an arrangement leads to the advantageous plastic deformation behavior.

Preferably, the first PEG component is selected from PEG 1000-8000, e.g. PEG 1000, PEG 1500, PEG 3000, PEG 3350, PEG 4000, PEG 6000 or PEG 8000; and the second PEG component comprised in the composition is selected from PEG 200-600, e.g. PEG 200, PEG 300, PEG 400, PEG 500 or PEG 600.

For example, PEG 300 and PEG 1500, PEG 400 and PEG 3350, or PEG 400 and PEG 4000 may be combined. Even more preferably, the composition comprises (exactly) two distinct PEG components, wherein the first PEG component is PEG 1500 and the second PEG component is PEG 300.

In the compositions of the inventions, the ratio (w/w) between (i) the first PEG component, in particular the PEG component having a solid physical state at 25° C., such as a PEG selected from PEG 1000-8000, and (2) the second PEG component, in particular the PEG component having a liquid physical state at 25° C., such as a PEG selected from PEG 200-600 may be from 1:10 to 10:1, preferably from 1:3 to 3:1, more preferably from 1:2 to 2:1, even more preferably from 1:1.75 to 1.75:1, and still more preferably from 1:1.5 to 1.5:1.

In some embodiments, the amount (weight) of the first PEG component, in particular the PEG component having a solid physical state at 25° C., such as a PEG selected from PEG 1000-8000, is about the same as the amount (weight) of the second PEG component, in particular the PEG component having a liquid physical state at 25° C., such as a PEG selected from PEG 200-600. For example, the ratio (w/w) between (i) the first PEG component, in particular the PEG component having a solid physical state at 25° C., such as a PEG selected from PEG 1000-8000, and (2) the second PEG component, in particular the PEG component having a liquid physical state at 25° C., such as a PEG selected from PEG 200-600, may be from 1:1.35 to 1.35:1, e.g. 1:1.

In some embodiments, the amount (weight) of the first PEG component, in particular the PEG component having a solid physical state at 25° C., such as a PEG selected from PEG 1000-8000, is higher than the amount (weight) of the second PEG component, in particular the PEG component having a liquid physical state at 25° C., such as a PEG selected from PEG 200-600. For example, the ratio (w/w) between (i) the first PEG component, in particular the PEG component having a solid physical state at 25° C., such as a PEG selected from PEG 1000-8000, and (2) the second PEG component, in particular the PEG component having a liquid physical state at 25° C., such as a PEG selected from PEG 200-600, is preferably from 1.1:1 to 3:1, more preferably from 1.1:1 to 2:1, even more preferably from 1.1:1 to 1.75:1, and still more preferably from 1.1:1 to 1.5:1.

Preferably, the amount (weight) of the first PEG component, in particular the PEG component having a solid physical state at 25° C., such as a PEG selected from PEG 1000-8000, is (the same or) lower than the amount (weight) of the second PEG component, in particular the PEG component having a liquid physical state at 25° C., such as a PEG selected from PEG 200-600. Accordingly, it is preferred that the ratio (w/w) between (i) the first PEG component, in particular the PEG component having a solid physical state at 25° C., such as a PEG selected from PEG 1000-8000, and (2) the second PEG component, in particular the PEG component having a liquid physical state at 25° C., such as a PEG selected from PEG 200-600, is preferably from 1:3 to 1:1.1, more preferably from 1:2 to 1:1.1, even more preferably from 1:1.75 to 1:1.1, still more preferably from 1:1.5 to 1:1.1, and particularly preferably about 1:1.3575.

In some embodiments, PEG 1500 and PEG 300 may be combined in a ratio (PEG 1500:PEG 300) of 1:1 to 1:2, preferably in a ratio of 1:1 to 1:1.5, e.g. 1:1.3575. In another example, PEG 3350 and PEG 400 may be combined in a ratio (PEG 3350:PEG 400) of 1:1 to 1:2, preferably in a ratio of 1:1 to 1:1.5, e.g. 1:1.3575. In another example, PEG 4000 and PEG 400 may be combined in a ratio (PEG 4000:PEG 400) of 1:1 to 1:2, preferably in a ratio of 1:1 to 1:1.5, e.g. 1:1.3575. Particularly preferably, PEG 1500 and PEG 300 are combined in a ratio (PEG 1500:PEG 300) of 1:1 to 1:1.5, e.g. 1:1.3575.

In addition, the compositions of the invention usually include DMSO. The present inventors have found that aqueous solvents fail to provide sufficient stability for decitabine or azacitidine. Among different non-aqueous solvents, only DMSO showed sufficient solubility for relevant dosage strengths. DMSO thus advantageously acts as non-aqueous solvent for decitabine or azacitidine. In some embodiments, DMSO may be combined with other non-aqueous, polar solvents, in particular with propylene glycol, e.g. at a ratio of 1:1. In other embodiments, the composition does not comprise further solvents other than DMSO (and PEG).

In general, the composition may advantageously contain sufficient DMSO to provide solubility at the envisaged decitabine or azacitidine concentration, while (large) excess amounts of DMSO should be avoided. Accordingly, the final concentration of DMSO may depend on the concentration of decitabine or azacitidine (or the salt thereof) in the composition. In view of the envisaged concentration of decitabine or azacitidine (or the salt thereof) in the composition, the DMSO concentration may be determined. The solubility of decitabine in DMSO is approximately 46 mg/ml.

In some embodiments, the composition comprises DMSO at a concentration (weight by weight (w/w)) of 1%-60%, such as 1-55%, 1-50%, 1-45%, 1-40%, 1-35%, 1-30%, 1-25% or 1-20%. Preferably the composition comprises DMSO at a concentration (weight by weight (w/w)) of 1%-15%, more preferably 2%-10%, even more preferably 4%-7%, still more preferably 5%-6%, and particularly preferably about 5.7%. While the present inventors found that such a concentration of DMSO is particularly advantageous for topical formulations (compositions) of the invention containing about 0.1 mM to 10 mM decitabine or azacitidine (or the salt thereof), higher concentrations, i.e. more than 10 mM decitabine or azacitidine (or the salt thereof), may require increasing concentrations of DMSO in the final composition owing to the solubility of decitabine (approximately 46 mg/ml) in DMSO. In such a case of higher DMSO concentrations, the concentration of the liquid PEG component(s) in the composition may be reduced to compensate for an increasing liquidity of the drug product formulation with higher concentrations of DMSO in the final composition.

Preferably, the compositions of the invention are essentially free of water. For example, the composition may contain less than 10% (w/w) water, preferably less than 5% water, more preferably less than 3% or 4% water, more preferably less than 2% water, even more preferably less than 1% water and still more preferably less than 0.5% water. In general, the less water is contained in the composition, the more it is preferred. Ideally, the composition does not comprise water or does not comprise any water. As shown in the appended examples, aqueous compositions negatively affect the stability of decitabine in the composition.

Accordingly, aqueous solvents are preferably avoided. In some embodiments, the composition may contain less than 10% (w/w) of an aqueous solvent, preferably less than 5% of an aqueous solvent, more preferably less than 3% or 4% of an aqueous solvent, more preferably less than 2% of an aqueous solvent, even more preferably less than 1% of an aqueous solvent and still more preferably less than 0.5% of an aqueous solvent. In general, the fewer of an aqueous solvent is contained in the composition, the more it is preferred. Ideally, the composition does not comprise an aqueous solvent.

In some embodiments, polar protic solvents are preferably avoided. Protic solvent are solvents containing a labile H⁺, i.e. they can readily donate a proton. Protic solvents include, e.g., solvents having a hydrogen atom bound to an oxygen (as in a hydroxyl group), a nitrogen (as in an amine group), or fluoride (as in hydrogen fluoride). While the most common polar protic solvent is water, non-limiting examples of other common polar protic solvents, which may be avoided, include formic acid, n-butanol, isopropanol, nitromethane, ethanol, methanol and acetic acid. In some embodiments, the composition may contain less than 10% (w/w) of a polar protic solvent, preferably less than 5% of a polar protic solvent, more preferably less than 3% or 4% of a polar protic solvent, more preferably less than 2% of a polar protic solvent, even more preferably less than 1% of a polar protic solvent and still more preferably less than 0.5% of a polar protic solvent. In general, the fewer of a polar protic solvent is contained in the composition, the more it is preferred. Ideally, the composition does not comprise a polar protic solvent. For example, the composition may be essentially free of ethanol. In some embodiments, the composition may contain less than 10% (w/w) ethanol, preferably less than 5% ethanol, more preferably less than 3% or 4% ethanol, more preferably less than 2% ethanol, even more preferably less than 1% ethanol and still more preferably less than 0.5% ethanol. In general, the fewer ethanol is contained in the composition, the more it is preferred. Ideally, the composition does not comprise ethanol.

In some embodiments, the compositions of the invention are essentially free of glycerin. For example, the composition may contain less than 10% (w/w) glycerin, preferably less than 5% glycerin, more preferably less than 3% or 4% glycerin, more preferably less than 2% glycerin, even more preferably less than 1% glycerin and still more preferably less than 0.5% glycerin. In general, the fewer glycerin is contained in the composition, the more it is preferred. Ideally, the composition does not comprise glycerin.

Preferably, the composition is essentially homogenous. For example, the PEG included in the composition is preferably not in the form of PEGylated liposomes. In some embodiments, the composition is (essentially) free of hormones. In particular, the composition does not comprise thyroid stimulating hormone (TSH).

In some embodiments, the composition of the invention does not correspond to a composition, which comprises glycerin. In some embodiments, the composition of the invention does not correspond to a composition which comprises polypropylene glycol. In some embodiments, the composition of the invention does not correspond to a composition, which comprises glycerin and polypropylene glycol. In some embodiments, the composition of the invention does not correspond to a composition, which comprises a combination of glycerin, polypropylene glycol and PEG.

In some embodiments, the composition of the invention does not contain an acidifying agent.

In some embodiments, the composition of the invention does not correspond to a composition comprising any of the following components selected from the group consisting of a stabilizing agent (other than PEG), an acidifying agent, a buffer salt, a pH modifier, a drying agent, and an antioxidant or a mixture thereof.

In some embodiments, the composition of the invention does not comprise any one of the following components, selected from the group consisting of a stabilizing agent other than PEG, an acidifying agent, a buffer salt, a pH modifier, a drying agent, an antioxidant, glycerin, and polypropylene glycol or a mixture thereof.

In some embodiments, the composition of the invention (essentially) consists of decitabine or azacitidine (or the salt thereof), PEG and DMSO. In other words, it is preferred that the composition does not comprise any component other than decitabine or azacitidine (or the salt thereof), PEG and DMSO. The present inventors have found that no further components are required to formulate a composition, which can be easily applied topically (specifically to a target area), which is effective for treating human papillomavirus (HPV)-related pre-cancerous conditions, and which provides sufficient stability of the active ingredient (decitabine or azacitidine (or the salt thereof)).

In some embodiments, the composition of the invention comprises decitabine or azacitidine (or the salt thereof), PEG 1500, PEG 300 and DMSO. In particular, the composition of the invention may consist of decitabine or azacitidine (or the salt thereof), PEG 1500, PEG 300 and DMSO.

In some embodiments, the composition comprises (or consists of):
0.1-10 mM (0.0228 mg/g-2.28 mg/g) decitabine (or azacitidine),
about 5.7% (w/w) DMSO,
about 40% (w/w) PEG 1500, and
about 54-55% (w/w) PEG 300.

The composition of the present invention may preferably exhibit stability of its active ingredient decitabine or azacitidine over an extended period of time. Degradation of the active ingredient is prevented by the inventive composition such that more than 90%, or more than 93% or more than 94% of the initially formulated active ingredient may be preferably maintained for a period of at least 3 months, in particular at storage conditions at 5° C.+/−3° C. and/or at 25° C.+/−2° C. The stability may e.g. be determined by HPLC methods.

### Medical Treatment and Uses

The compositions of the invention may be for use in medicine, preferably wherein the pharmaceutical composition is applied topically. Non-limiting examples of medical conditions, which may be treated with the composition of the invention include medical conditions or disorders in the keratinizing and non-keratinizing epithelium/skin (e.g., that are accessible for topical treatment). The composition may thus be topically applied on non-keratinizing or keratinizing epithelium or a transition zone from non-keratinizing to keratinizing epithelium. Preferably, the composition of the invention is for use in the treatment of papillomavirus (PV)-related pre-cancerous conditions, preferably wherein the pharmaceutical composition is applied topically, for example to a lesion and/or to a genital area (of an animal or human patient). For example, the composition of the invention may be used for the treatment of animals infected by species-specific types of (human) papillomaviruses, such as equine sarcoids in horses that may be caused by bovine papillomaviruses. Preferably, the composition of the invention is for use in the treatment of human papillomavirus (HPV)-related pre-cancerous conditions, preferably wherein the pharmaceutical composition is applied topically, for example to a lesion and/or to a genital area (of a human patient).

Accordingly, the present invention also provides a method for the treatment of a medical condition in the keratinizing and non-keratinizing epithelium/skin, such as a papillomavirus (PV)-related medical condition in the keratinizing and non-keratinizing epithelium/skin or for reducing the risk of cancer comprising administering (an effective amount of) the composition of the invention as described above to a subject in need thereof. In some embodiments, the method is for the treatment of a human papillomavirus (HPV)-related pre-cancerous condition. In some embodiments, the composition is applied topically, for example to a lesion and/or to a genital area (e.g., of an animal or human patient).

The present invention also provides the use of the composition of the invention as described above for the manufacture of a medicament for the treatment of a medical condition in the keratinizing and non-keratinizing epithelium/skin, such as a papillomavirus (PV)-related medical condition in the keratinizing and non-keratinizing epithelium/skin, e.g. a human papillomavirus (HPV)-related pre-cancerous condition.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. Preferably, the subject or patient is a human.

Human papillomavirus (HPV), in particular high risk HPVs such as HPV 16, 18, 31, 33, 35, 39, 45, 51, 52, 56, 58, 59, and potentially 66 and 82, can cause cancer. HPV-related cancers (and respective pre-cancerous conditions) typically occur in parts of the body, where HPV infects epithelial cells, such as in the uterine cervix, oropharynx, anus, penis, vagina, and vulva. Accordingly, HPV-related pre-cancerous conditions include cervical pre-cancerous conditions (which may develop into cervical cancer), oropharyngeal pre-cancerous conditions (which may develop into oropharyngeal cancer), anal pre-cancerous conditions (which may develop into anal cancer), penile pre-cancerous conditions (which may develop into penile cancer), vaginal pre-cancerous conditions (which may develop into vaginal cancer), and vulvar pre-cancerous conditions (which may develop into vulvar cancer). Often, HPV infects squamous epithelial cells and, therefore, most HPV-related pre-cancerous conditions and cancers are squamous cell pre-cancerous conditions and squamous cell carcinoma, respectively. However, certain HPV-related (in particular cervical) pre-cancerous conditions and cancers may be adenocarcinomas or respective pre-cancerous conditions.

As used herein, the term “pre-cancerous” (also referred to as “premalignant”) refers to a condition, which may (or is likely to) develop into cancer. Treatment of a pre-cancerous condition typically includes reducing the risk of a development of the pre-cancerous condition into a cancer. HPV-related pre-cancerous conditions may be identified, for example, by cytology-based screening, such as a Pap test or Pap smear (in particular for cervical cancer screening). There are different pre-cancerous stages, which may be classified according to the respective healthcare systems. For example, the Bethesda System divides squamous cell abnormalities into the following categories, ranging from the mildest to the most severe: atypical squamous cells (ASC) with two subgroups, ASC-US (undetermined significance) and ASC-H (a high-grade squamous intraepithelial lesion cannot be excluded); low-grade squamous intraepithelial lesions (LSILs); high-grade squamous intraepithelial lesions (HSILs), and squamous cell carcinoma. Glandular cell abnormalities are divided in the Bethesda system into the following categories: atypical glandular cells (AGC), endocervical adenocarcinoma in situ (AIS), and adenocarcinoma. Of these stages, squamous cell carcinoma and adenocarcinoma are cancer, while the other (preceding) stages are pre-cancerous conditions.

As described above, the composition of the invention is preferably applied topically to a target area, e.g. a pre-cancerous lesion or an area shown or suspected to be affected by a pre-cancerous condition. The target area may be an anogenital or genital area or any other body part affected by HPV-related pre-cancerous condition, in particular the vulva. Examples of body parts, to which the composition of the invention may be applied topically include all body parts, where HPV infects cells, such as the cervix, oropharynx, anal canal, penis, vagina, and vulva. Thus, the composition may be applied to air-exposed body surfaces or to non-air-exposed surfaces (i.e. body cavities).

In some embodiment, the medical condition or disorder to be treated by the inventive composition refers to pre-cancerous lesions of the vulva, more specifically to vulvar intraepithelial neoplasia grade 2/3.

In some embodiments, a pre-cancerous condition may be identified during colposcopy and the composition of the invention may be applied, e.g. to a cervical region, for example during colposcopy.

In a specific embodiment, the composition of the invention may be used for the treatment of HPV-induced pre-cancerous lesions. Thereby, treatment of HPV-associated cancers is preferably excluded.

Advantageously, the method of treatment according to the present invention does not induce epithelial disintegration or ulceration of normal tissue surrounding the treated target lesion/diseased tissue.

Administration may be carried out by a gloved finger or with the help of a medical device, such as an applicator, a spatula, or a cervical cap.

The administration regimen may be designed as follows: Within a treatment cycle, the composition is administered at least 5 times, e.g. 5 to 15 applications. The administration may be carried for each treatment cycle e.g. once or twice per day or once every second or third day. The total treatment period may comprise at least 2 treatment cycles, such as 2 to 5 treatment cycles over a period of at least 2 weeks, e.g. 2 to 8 weeks. The composition preferably comprises 1 mM to 1 M of the active ingredient, preferably 1 mM to 50 mM. Per application, an amount of 5 to 500 mg, preferably 50 to 250 mg, of the formulation is applied to the affected tissue/lesion.

### Package for the Composition

Another aspect of the present invention is a package for the composition of the present invention. In an embodiment, the inventive composition is packaged in a primary container that contains a volume from 0.05 to 50 ml. Preferably, the primary packaging of the formulation allows for repetitive removal/withdrawal of the formulation from the container (such as a tube or any other suitable applicator or container for patient convenience as well as for facilitated transport and storage. A primary container designed as a tube may be preferable, which may be deformable or not deformable. The tube may be made of a plastic material or a metal or alloy material, e.g. of aluminum. Preferably, deformable (aluminum) tubes are employed for preventing the absorption of humid air into the tube. More specifically, the tube outlet may be equipped with an applicator, at least when being used, to allow removal of a defined amount of formulation by the strand length.

In another embodiment, the formulation may be presented in single-use primary packaging containers (such as a sachet).

The primary packaging may preferably contain a protective inner lacquer (e.g. when applying tubes) or another protective inner layer.

Alternatively, the composition may be provided as a capsule, the capsule containing the composition of the invention. The capsule may be preferably be composed of a self-dissolving material delivering the composition of the invention upon administration, e.g. upon insertion in the anogenital area. Other embodiments allowing packaging the inventive composition refer to (bioadhesive) patches or tampons. The iiinventive composition, preferably as an ointment, may be applied to such patches or tampons.

### Method for Preparing the Composition

The present invention also provides a method for preparing the pharmaceutical composition according to anyone of the previous claims, the method comprising the following steps:
(1) preparing a solution of decitabine or azacitidine (or the salt thereof) in DMSO;
(2) melting the PEG component(s) to obtain a homogeneous composition; and
(3) mixing the decitabine (or azacitidine) solution of step (1) with the melted PEG component(s) of step (2) to obtain a homogenous composition.

While step (3) follows steps (1) and (2), steps (1) and (2) may be performed in any order, for example step (1) before step (2) or step (2) before step (1) or steps (1) and (2) may be performed essentially in parallel (simultaneously). Advantageously, decitabine or azacitidine is initially dissolved in DMSO and, thus not in PEG. Thereby, decitabine or azacitdine come in contact with PEG only after they have been previously dissolved in DMSO.

In some embodiments, step (1) is performed at 20° C.-30° C., preferably at 22° C.-28° C., more preferably at 23° C.-27° C., even more preferably at about 25° C.

In some embodiments, in step (1) a stock solution may be used and the appropriate amount of stock solution for the relevant decitabine concentration may be added to the remaining weight of DMSO in a suitable container, and mixed to obtain a homogenous solution. For example, the following concentrations may be used for the preparation of the decitabine stock solution in DMSO (depending on final dosage strength):
For 10 and 50 mM: 44 mg/ml;
For 1 mM: 4.4 mg/ml;
For 100 PM: 0.44 mg/ml;
For 10 PM: 0.044 mg/mL.

In other embodiments, the solution of decitabine or azacitidine (or the salt thereof) in DMSO is prepared in step (1) without using a stock solution.

If more than a single polyethylene glycol (PEG) component is used, the distinct PEG components may be combined in step (2) and melted, e.g. in a suitable container, at moderate product temperature, for example at 50-60° C., preferably at 51-59° C., more preferably at 52-58° C., and even more preferably at 65° C.±2° C. In some embodiments, melting of the PEG component(s) is performed under (continuous) stirring. As an example, the PEG components PEG 1500 and PEG 300 may be weighed as follows: 40% (w/w) PEG 1500 and 30-55% (w/w) PEG 300. Distinct PEG components to be combined may be provided in step (2) in the same container. The PEG component(s) may be melted in step (2), in particular under stirring, for example, at 50-60° C., preferably at 51-59° C., more preferably at 52-58° C., and even more preferably at 65° C.±2° C. (e.g., over a water bath).

Thereafter, the PEG component(s) may be (actively or passively) cooled down, e.g. to 30-45° C., preferably 32-40° C., more preferably to 36° C.±2° C., before step (3) starts.

In step (3), the decitabine (or azacitidine) solution of step (1) is mixed with the melted PEG components of step (2) to obtain a homogenous composition. To this end, the decitabine (or azacitidine) solution of step (1) may be transferred (added) to the melted polyethylene glycol mixture of step (2). To obtain a homogeneous mixture, it may be stirred for an appropriate time or mixed; e.g. on a lab test tube shaker (vortexer). Thereafter (when a homogenous composition is obtained), the composition may be (actively or passively) cooled down, e.g. to room temperature (about 25° C.), for example under stirring. In some embodiments, it may be optionally mixed thereafter again, e.g. with a pharmaceutical mixing system.

After step (3), the resulting composition may be packaged, for example filled into an appropriate container, such as an ointment tube.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 1 (A) the stability test of decitabine in an aqueous buffer (about 25° C., two independent samples) and (B) the degradation products of decitabine in aqueous buffer (about 25° C.).

FIG. 2 shows for Example 1 the stability of decitabine in DMSO at 2-8° C. in the left (lower) panel and at room temperature (about 25° C.) in the right (upper) panel.

FIG. 3 shows for Example 1 the stability of decitabine in DMSO in a polyethylene glycol-based formulation (1 mM) at 2-8° C. in the left (lower) panel and of decitabine in DMSO in a glycerol/carbopol-based formulation (1 mM) at 2-8° C. in the right (upper) panel.

FIG. 4 shows for Example 1 the stability of decitabine in DMSO in a polyethylene glycol-based formulation (1 mM) at different temperatures. “Starting value”=concentration at the day after preparation; “room temperature”: about 25° C.; quantitative values: 1099.4 μM/1110.8 μM/1048.9 M.

FIG. 5 shows for Example 1 the stability of an exemplary decitabine formulation containing 2.28 mg/g (10 mM, nominal concentration) decitabine over three months of storage at different temperatures as indicated. The percentage value indicates the actual relative to the nominal decitabine content.

FIG. 6 shows for Example 2 an overlay of the flow profiles of PEG-based Placebo formulation and PEG-based 10 mM decitabine formulation.

FIG. 7 shows for Example 2 the viscosity at constant rate 1.0 sec⁻¹ as function of time (t) for PEG-based Placebo formulation.

FIG. 8 shows for Example 2 an Overlay LVER of PEG-based Placebo formulation and PEG-based 10 mM decitabine formulation.

FIG. 9 shows for Example 3 the results from a 2-week topical treatment experiment on organotypic cultures (OTC) consisting of keratinocytes and HPV-transformed SiHa cells grown on dermal equivalents that were treated with the polyethylene glycol (PEG)-based Decitabine-containing formulation at different strengths [0.1 mM, 1 mM or 10 mM] or a placebo formulation as control. Representative areas of tumor nests and adjacent normal epithelium from whole-slide scans of hematoxylin and eosin (HE)-stained tissue sections from one biological replicate per treatment condition are shown at 10-fold (A1-D1) and 40-fold (A2-D2) magnification. Black lining indicates areas of tumor nests consisting of HPV-transformed SiHa cells, dotted grey lining indicates stratifying normal epithelial cells, black rectangles within images A1-D1 indicate tissue areas that are shown in greater detail at 40× magnification in the respective images A2-D2.

FIG. 10 shows for Example 5 the p16^{INK4a} immunohistochemical staining results from a 2-week (A, B) and 4-week (C, D) topical treatment experiment with a PEG-based Decitabine-containing formulation [1 mM]/matching Placebo formulation on ex vivo cultivated HPV-induced pre-cancerous lesions from the vulva at 20-fold magnification of whole-slide scans.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Stability of Decitabine in Different Formulations

To assess the stability of decitabine in different formulations, HPLC analysis was used as follows:

The HPLC method was based on a previously published method by Rogstad, D. et al. Chemical decomposition of 5-aza-2′-deoxycytidine (Decitabine): kinetic analyses and identification of products by NMR, HPLC, and mass spectrometry. Chem Res Toxicol 2009; 22 (6): 1194-204. The parameter/values used are shown in Table 1 below.

TABLE 1

| Parameter | Value | |
|---|---|---|
| HPLC | Jasco HPLC 2080 (PU-2080Plus, LG-2080-02S, | |
| | DG-2080-53) with Jasco AS-1555 Autosampler, | |
| | Merck T-6300 Column Thermostat, and | |
| | Shimadzu SPD-10A UV-VIS Detector | |
| Column | Supelcosil LC-18-S (RP C-18 250 × 4.6 | |
| | mm 5 μm)with 20 × 4.0 mm Supelcosil | |
| | LC-18 5 μm guard column | |
| Mobile phase | 20 mM ammonium acetate pH 6.8 with | |
| | 2% methanol | |
| Flow rate | 1 | mL/min |
| Column | 25° | C. |
| temperature | | |
| Detection | UV absorption 240 nm | |
| Injection volume | 20 | μL |

| | |
|---|---|
| Internal | 2′-desoxyuridine in DMSO |
| standard stock | (1 mg/mL) |
| Sample solution | Dissolving of approx. 180 mg formulation in |
| | 500 μL internal standard stock in DMSO; |
| | Vortexed until completely dissolved |

### Steps:

1. Mixing of 200 μl sample solution with 444 μL mobile phase
2. Immediate Injection
3. Quantification using internal standard and separately recorded internal standard calibration curve (constant internal standard concentration & increasing decitabine concentration)
4. Identification of peaks as described in Rogstad, D. et al. Chemical decomposition of 5-aza-2′-deoxycytidine (Decitabine): kinetic analyses and identification of products by NMR, HPLC, and mass spectrometry. Chem Res Toxicol 2009; 22 (6): 1194-204.

### A. Stability of Decitabine in Aqueous Media

To test the stability of β-decitabine in an aqueous medium, two stock solutions of β-decitabine in DMSO were prepared and mixed 1:2 with the HPLC mobile phase (20 mM ammonium acetate pH 6.8). The samples were stored at room temperature (about 25° C.) and their content of β-decitabine, which is considered to represent the active anomer of decitabine, was measured for up to 14 days.

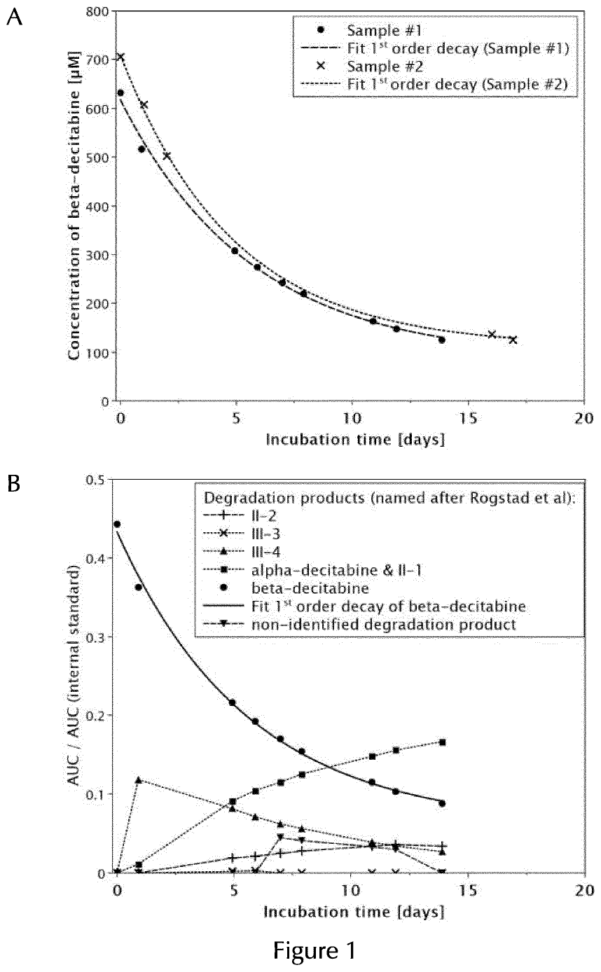

Results are shown in FIG. 1. FIG. 1A shows the decrease in β-decitabine over the observed time period, which is well described by a 1st order decay function. One of the samples was further analyzed in terms of the appearance of known degradation products of β-decitabine. The results can be seen in FIG. 1B. These results clearly show that an aqueous medium cannot be used due to the observed instability of decitabine in the aqueous medium.

In summary, these data show that water-containing formulations are not suitable for the decitabine formulation due to the rapid degradation of decitabine in aqueous environments.

### B. Solubility of Decitabine in Different Non-Aqueous Media

Due to the unsuitability of aqueous media, different non-aqueous media were tested with regard to the solubility of decitabine in the non-aqueous media. As decitabine is a nucleoside, several polar, water-free solvents, that would be well suited for further formulation experiments, were selected to determine the solubility of decitabine. However, of the four tested solvents-glycerol, PEG 300, absolute ethanol, and DMSO-only DMSO showed sufficient solubility for relevant dosage strengths. Therefore, the DMSO stock was chosen as the next candidate for stability and formulation testing.

### C. Long-Term Stability of Decitabine in DMSO

A 3 mM stock solution of decitabine in DMSO was prepared and one aliquot each was stored at room temperature and at 2-8° C. Its β-decitabine concentration was determined by HPLC for almost three months.

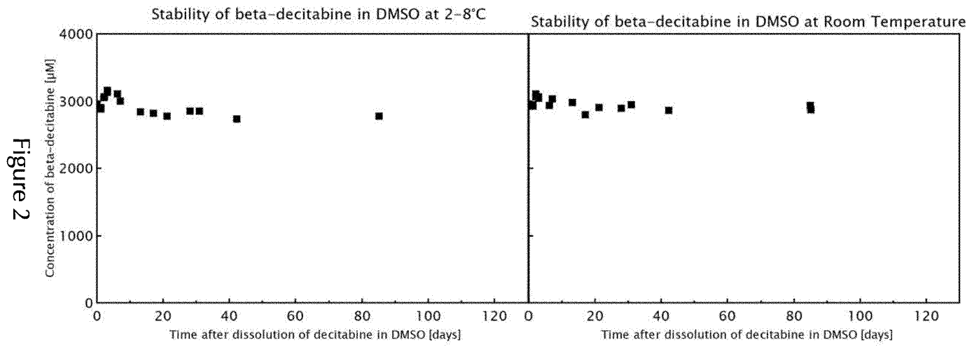

The results are shown in FIG. 2. These tests showed almost no change in the β-decitabine concentration, and even though the sample size of these first tests was too low to reach statistical significance, sufficient long-term stability was observed, especially when compared to the (in) stability in aqueous buffer.

### D. Long Term Stability of Formulation Candidates

Previous tests of formulations without decitabine resulted in the selection of two formulations that showed appropriate viscosity for handling and application by the patient and that were also compatible with the DMSO concentrations needed for the relevant dosage strengths of β-decitabine:
(1) a formulation based on a mix of polyethylene glycols; and
(2) a glycerol-based gel.

Therefore, first batches of these formulations were prepared for the first in vitro tests as follows:

### (1) Polyethylene Glycol (PEG) Formulation

Briefly, the following steps were performed:
1. The polyethylene glycol (PEG) components were combined and melted in a suitable container under continuous stirring at moderate product temperature (50-60° C.).
2. A stock solution of decitabine in DMSO was prepared. The appropriate amount of stock solution for the relevant decitabine concentration was added to the remaining weight of DMSO in a suitable container, and mixed to obtain a homogenous solution.
3. The decitabine solution in DMSO was transferred to the melted polyethylene glycol mixture of step 1 and stirred for an appropriate time to obtain a homogeneous mixture.

In more detail, the exemplified formulations were obtained as follows:
1. Preparation of the decitabine stock solution in DMSO depending on final dosage strength:
   For 10 and 50 mM: 44 mg/ml;
   For 1 mM: 4.4 mg/ml;
   For 100 μM: 0.44 mg/ml;
   For 10 μM: 0.044 mg/ml
2. Weigh polyethylene glycol compounds in 50 mL centrifuge tube: 40% (m/m) PEG 1500 60-x % (m/m) PEG 300 (x % (m/m)=DMSO stock (approx. 5.7% for dosage strengths up to 10 mM, see below)
3. Melt over water bath (50-60° C.)
4. Add appropriate amount of DMSO stock solution (see above) to melted PEG components
5. Mix on lab test tube shaker (vortexer)
6. Transfer melted batch to Topitec jar
7. Let cool down
8. Mix with Topitec (Level 1, 60s)
9. Fill into ointment tube
10. Close packaging (close tube and shrink-wrap)

Thereby, the following compositions were obtained (Table 2):

| | 10 μM | 100 μM | 1 mM | 10 mM | 50 mM |
|---|---|---|---|---|---|
| Decitabine [m/m] | 0.0002% | 0.002% | 0.02% | 0.23% | 1.14% |
| DMSO [m/m] | 5.7% | 5.7% | 5.7% | 5.7% | 28.5% |
| PEG 300 [m/m] | 54.3% | 54.3% | 54.3% | 54.1% | 30.4% |
| PEG 1500 [m/m] | 40.0% | 40.0% | 40.0% | 40.0% | 40.0% |

### (2) Glycerol Gel Formulation

1. Preparation of the decitabine stock solution in DMSO.
   For 10 and 50 mM: 44 mg/ml;
   For 1 mM: 4.4 mg/ml;
   For 100 μM: 0.44 mg/mL;
   For 10 PM: 0.044 mg/mL.
2. Pipette glycerol in Topitec jar
3. Add decitabine stock solution (5.8% m/m of final mass)
4. Add Carbopol® 974P (0.5% m/m of glycerol mass)
5. Immediately mix with Topitec
6. Fill into ointment tube

Thereby, the following compositions were obtained (Table 3):

| | 10 μM | 100 μM | 1 mM | 10 mM | 50 mM |
|---|---|---|---|---|---|
| Decitabine [m/m] | 0.0002% | 0.002% | 0.02% | 0.23% | 1.15% |
| DMSO [m/m] | 5.8% | 5.8% | 5.8% | 5.8% | 27.8% |
| Glycerol [m/m] | 93.7% | 93.7% | 93.7% | 93.5% | 69.8% |
| Carbopol ® 974P | 0.5% | 0.5% | 0.5% | 0.5% | 1.3% |
| [m/m] | | | | | |

Note:
A higher Carbopol ® concentration was used for dosage strengths >10 mM.

Aliquots of these batches were stored at 2-8° C. to evaluate their long-term stability.

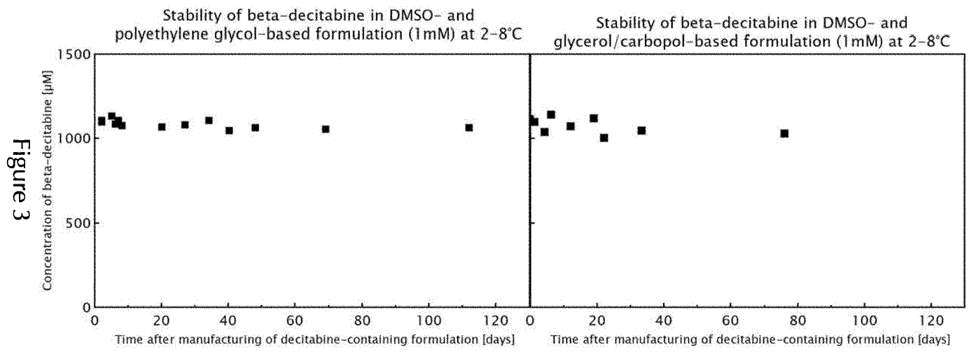

Results are shown in FIG. 3. FIG. 3 shows the β-decitabine content of the samples quantified at up to 112 days (PEG) and 76 days (glycerol), respectively. Again, even though the sample size of these first tests was too low to reach statistical significance, sufficient long-term stability could be observed.

### E. Stability Test at Different Temperatures

The stability of the PEG-based formulation as described above was then tested at different temperatures.

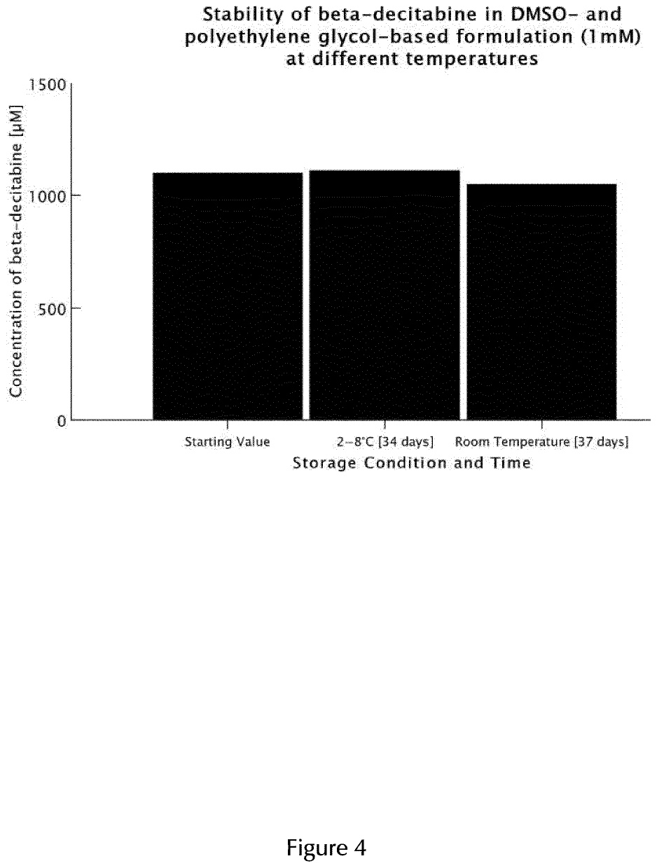

First tests are shown in FIG. 4. FIG. 4 shows the β-decitabine content of three aliquots of the same production batch of the PEG-based formulation: the left bar is the content the day after preparation, the center bar is the content after about a month at 2-8° C., and the right bar shows an aliquot that was not stored under controlled conditions but rather left at room temperature after using it for in vitro experiments and then quantified also after about a month.

As one can see, even at these uncontrolled conditions, no considerable degradation had taken place, suggesting the possibility of room temperature storage for a final developed drug product.

Subsequently manufactured batches at distinct strengths [0.1 mM, 1 mM, and 10 mM] essentially as described by Table 2 were stored under controlled conditions, namely at −80° C., −20° C., 5° C. and 25° C. Decitabine assay was determined by HPLC after manufacture (TO) as well as after one (T1), two (T2) and three (T3) months of storage at the respective conditions.

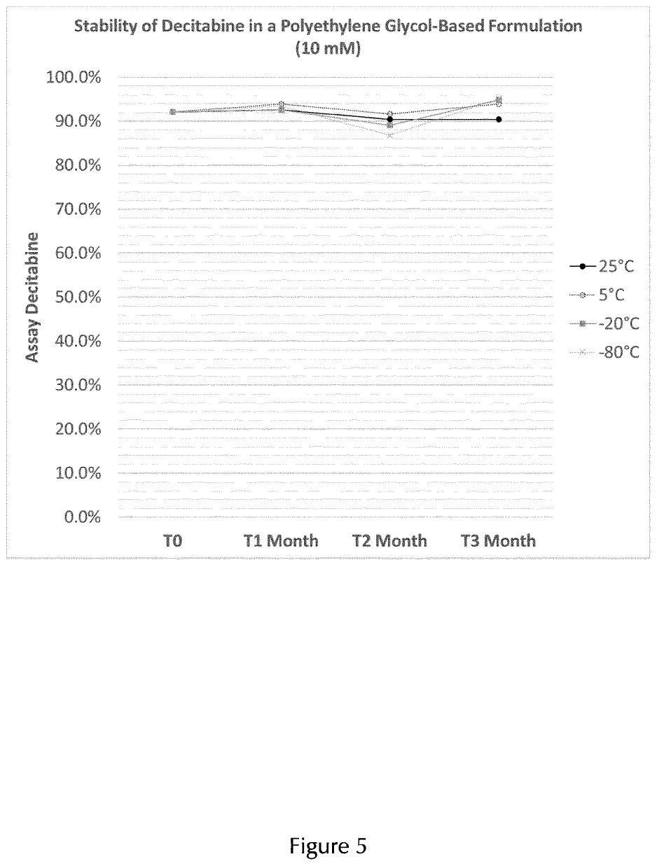

Results are shown in FIG. 5. Essentially no changes in the assay values were observed for storage at −80° C., −20° C., 5° C. or 25° C. in these preliminary analyses. Accordingly, the formulation is stable even for prolonged storage at room temperature (about 25° C.).

In summary, the data obtained in Example 1 showed the necessity of using a stock solution of decitabine in DMSO for the preparation, due to the lack of solubility in other usable media. The long-term stability data of decitabine in dimethylsulfoxide (DMSO) looked promising and two potential formulations were developed on the basis of DMSO-dissolved decitabine—a glycerol-based gel and a polyethylene-glycol based ointment. Both showed good long-term stability in first orientating tests and the polyethylene glycol based formulation was chosen for further development due to results from the in vitro tests not described here.

### Example 2: Assessment of Viscosity of the Formulation

For topical application, the viscosity of the composition should on the one hand be sufficiently high to substantially prevent running or unintentional spreading of the composition from the area where it is applied to other areas. On the other hand, the viscosity of the composition should be sufficiently low to enable the patient to readily remove the required dose of the composition from the container in which it is available (e.g. a tube or the like), apply it evenly over the affected area to ensure an even dosing of the active component, and to avoid or minimize friction at the site of application which could cause local irritation and/or pain.

In the following, examples for determining the viscosity of exemplified compositions are provided.

### A: Flow Profile Determination

The flow profile was determined for PEG-based Placebo formulation and PEG-based 10 mM decitabine formulation. PEG-based 10 mM decitabine formulation was prepared as described below:
(1) preparing a solution of decitabine or azacitidine (or the salt thereof) in DMSO at room temperature (about 25° C.);
(2) melting the PEG component(s), e.g. at 65° C.±2° C. under stirring to obtain a homogeneous composition, and then cooling down to 36° C.±2° C.;
(3) mixing the decitabine (or azacitidine) solution of step (1) with the melted PEG components of step (2) to obtain a homogenous composition; and
(4) cooling down to room temperature (about 25° C.) under stirring.

PEG-based placebo formulation was prepared as follows:
(1) melting the PEG component(s), e.g. at 65° C.±2° C. under stirring to obtain a homogeneous composition; and then cooling down to 36° C.±2° C.;
(2) addition of DMSO with the melted PEG components of step (1) to obtain a homogenous composition; and
(3) cooling down to room temperature (about 25° C.) under stirring.

Each sample was loaded at room temperature (25° C.) into the rheometer (Anton Paar: Model: MCR302); thermostated Bath (Viscotherm VT2 Anton Paar) with a gap of 398 nm (cone-plate geometry CP4-40 mm).

A logarithmic ascending and descending stress sweep was applied on the samples. The conditions are shown in Table 4 below.

TABLE 4

| | Instruments | Step |
|---|---|---|
| Mode | settings | duration |
| Loading samples & Rest Time | Temp: 25° C. | 300 sec. |
| | (+/−0.5° C.) | |
| Ascending logarithmic shear rate sweep | 0.01 to 100 sec⁻¹ | 201 sec. |
| Decreasing logarithmic shear rate sweep | 100 to 0.01 sec⁻¹ | 201 sec. |

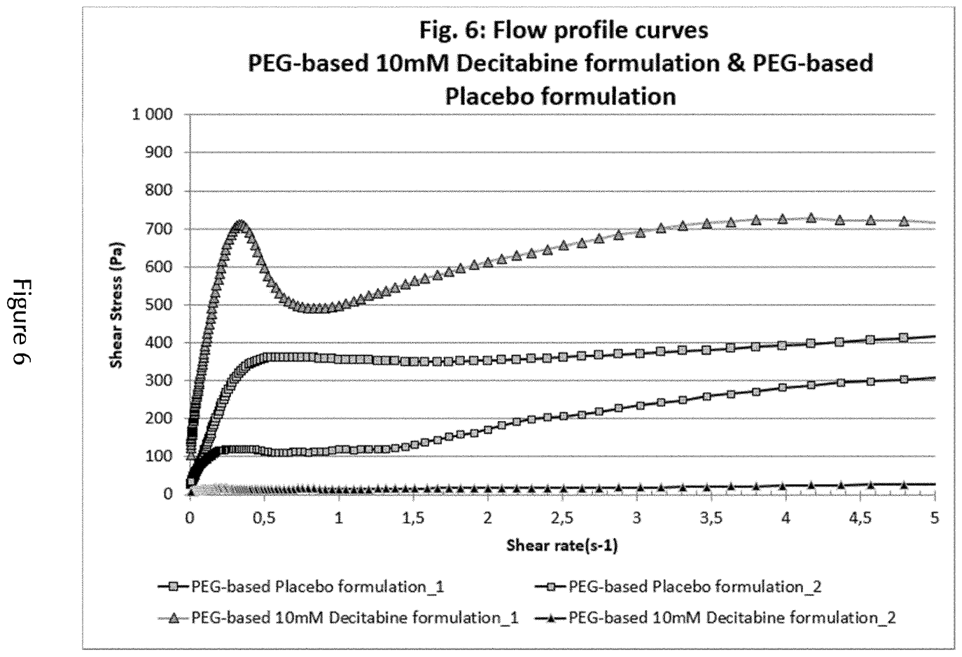

Results are shown in FIG. 6. Table 5 below shows viscosity values (in Pa·s) extracted at 1.0 sec⁻¹ on the ascending curve as well as yield point values (in Pa) extrapolated from the maximum of the curve on the beginning of the ascending curve:

TABLE 5

| | Viscosity at | Yield Point |
|---|---|---|
| | 1.0 sec⁻¹ | Extrapolation |
| Samples | (Pa · s) | (Pa) |
| PEG-based placebo formulation | 356 | 362 |
| PEG-based 10 mM decitabine | 498 | 711 |
| formulation | | |

The shape of the flowing curve in FIG. 6 indicates that both samples present a shear thinning behavior. The term “shear thinning” as used herein refers to the common characteristics of non-Newtonian fluids in which the fluid viscosity decreases with increasing shear rate or stress. Shear thinning is observed in suspensions, emulsions, polymer solutions and gels. Due to shear thinning attributes, decreasing the viscosity of an ointment is made possible by increasing the rate of shear. Basically as a result of the decrease in viscosity upon increase in shear rate the “shear thinning” property is a measure of the ability of the ointment structure to be temporarily deformed during its application by rubbing onto the skin.

At the beginning of the flowing curve shown in FIG. 6, the shear stress increases rapidly to a maximum value corresponding to the yield point. From this value the sample starts to flow. As shown both ascending and descending curves are not the same which reflects a thixotropic behavior.

In summary, the results show that the PEG-based 10 mM decitabine formulation is more viscous with a higher yield point compared to PEG-based Placebo formulation.

### B: Viscosity at Constant Rate

The placebo sample was loaded at room temperature (25° C.) into the rheometer (Anton Paar: Model: MCR302); thermostated Bath (Viscotherm VT2 Anton Paar) with a gap of 398 nm (cone-plate geometry CP4-40 mm).

A linear stress sweep was applied to the samples up to a targeted constant rate fixed at 1 s⁻¹ (1 sec⁻¹). After pre-shear for 300 seconds the viscosity was determined.

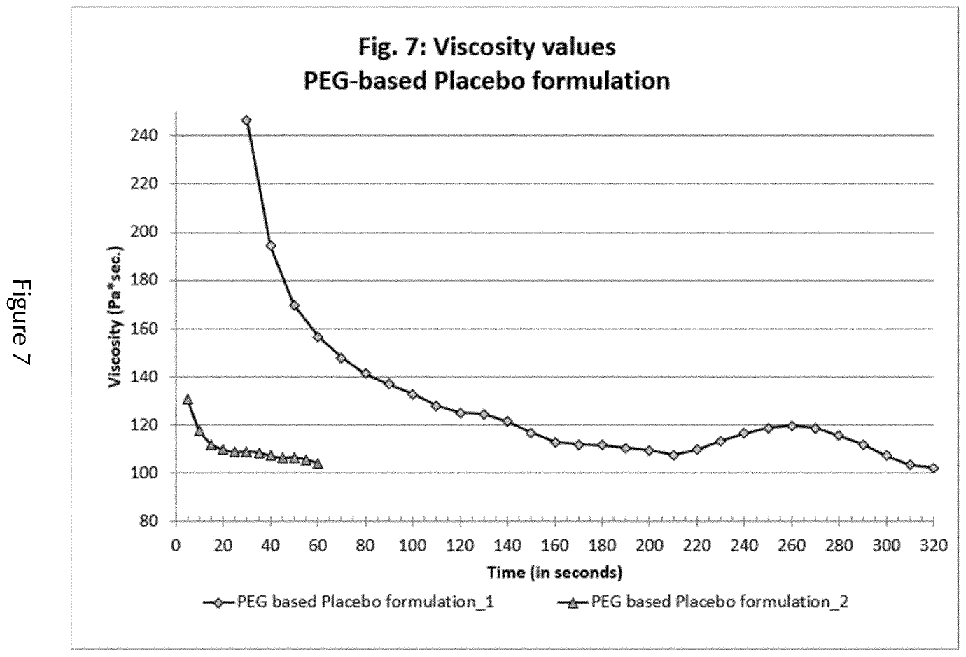

Results are shown in FIG. 7 and in Table 6 below:

TABLE 6

| | Viscosity at | Viscosity at the |
|---|---|---|
| | the end of | beginning of |
| | pre-shear | measurement |
| Sample | step (Pa · s) | (Pa · s) |
| PEG-based placebo | 102 | 131 |
| formulation | | |

At the beginning of the measurement the viscosity decreases rapidly and never stabilizes during the measurement. At the end of the pre-shear at constant rate the viscosity quickly increases from 102 Pa·s to 131 Pa·s. This observation confirms the thixotropic behavior of the sample which rapidly recovers part of its viscosity once the constraint is stopped. That means that the samples do not leak from the tube after pressure and from the affected areas of the skin to unaffected areas when the rubbing onto the skin is stopped.

### C: Determination of the Linear Viscoelastic Region (LVER) and the Complex Viscosity

Oscillatory measurements are useful for studying processes involving a change of structure in the sample. The strain has to remain small enough, not to disturb the ongoing physical or chemical process. A stress sweep test (i.e., measurements of elastic and loss moduli as function of stress at fixed frequency) was performed to confirm the shear-thinning character of the samples.

Each sample (as described in A) was loaded at room temperature (25° C.) into the rheometer (Anton Paar: Model: MCR302); thermostated Bath (Viscotherm VT2 Anton Paar) with a gap of 398 nm (cone-plate geometry CP4-40 mm).

### Step 1: Determination of the Linear Viscoelastic Region (LVER)

TABLE 7

| | | Step |
|---|---|---|
| Mode | Instrument settings | duration |
| Loading samples & Rest Time | Temp: 25° C. | 300 sec. |
| | (+/−0.5° C.) | |
| Ascending logarithmic sweep | 0.01 to 1 000 Pa | 602 sec. |

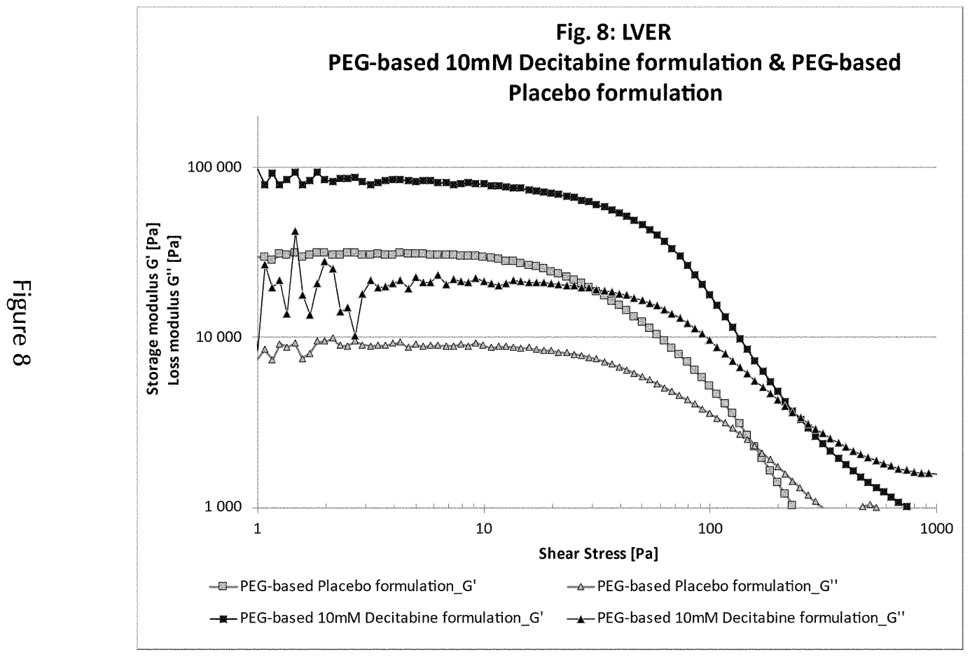

Results are shown in FIG. 8 and in Table 8 below:

TABLE 8

| | | | | | Shear stress |
|---|---|---|---|---|---|
| | | | | | value at |
| | Elastic | Loss | | Length of | cross point |
| | Modulus | modulus | Tangent | LVER | G′ = G″ |
| Samples | G′ (Pa) | G″ (Pa) | delta | (Pa) | (Pa) |
| PEG-based | 30 501 | 8 962 | 0.294 | 14.7 | 158 |
| placebo | | | | | |
| formulation | | | | | |
| PEG-based | 82 394 | 20 450 | 0.249 | 15.8 | 251 |
| 10 mM | | | | | |
| decitabine | | | | | |
| formulation | | | | | |

The term “elastic modulus” (also referred to as “Young's modulus” or the storage modulus (G′) is defined herein as the change in stress with an applied strain (that is the ratio of shear stress (force per unit area) to the shear strain (proportional deformation)) in a material. Essentially the elastic modulus is a quantitative measurement of stiffness of an elastic material that measures the ability of the tested material to return to its original shape and size.

The term “loss modulus” (also referred to as viscous modulus (G″)) characterizes the viscous portion of the viscoelastic behavior, which can be seen as the liquid-state behavior of the sample. It characterizes the deformation energy lost (dissipated) through internal friction when flowing. Viscoelastic solids with G′>G″ have a higher elastic modulus than loss modulus.

The measure of the elastic modulus and the loss modulus are reported as the force per unit area (N/m²) or the pascal (Pa) in which one pascal is equivalent to one Newton (1N) of force applied over an area of one meter squared (1 m²)). This pascal unit is an art-recognized term often used to define a unit of pressure, tensile strength, stress and elasticity.

For both samples (PEG-based placebo formulation and PEG-based 10 mM decitabine formulation) the linear viscoelastic region goes from 1 to around 15 Pa of oscillatory constraint. Over 15 Pa both moduli G′ and G″ continuously decrease. Shear-thinning behavior is observed (i.e., decrease of elastic modulus G′ as a function of the applied stress) at higher stress values.

The term “loss of tangent tan 8” or “tan 8” refers herein to the tangent of the phase angle that is the ratio of viscous modulus (G″) to elastic modulus (G′) and a helpful quantifier of the presence and the degree of elasticity in a fluid. The tan 8 values of less than unity indicate elastic-dominant (i.e. solid-like) behavior and values greater than unity indicate viscous-dominant (i.e. liquid-like) behavior.

For both samples tan 8 is less than unity which confirms the elastic-dominant behavior.

### Step 2: Determination of the Complex Viscosity

The complex viscosity measurements were performed in linear viscoelastic range of deformation at a predetermined strain amplitude fixed at 5 Pa and fixed frequency at 1 Hz in order to not disturb, or limit, the structural changes.

TABLE 9

| Mode | Instruments settings | Step duration | |
|---|---|---|---|
| Loading samples | Temp: 25° C. | 300 | sec. |
| & Rest Time | (+/−0.5° C.) | | |
| Pre-shear | 5.0 Pa | 60 | sec. |
| Measurement | 5.0 Pa | 60 | sec. |

Results are shown in Table 10 below:

TABLE 10

| | Complex viscosity | Average complex |
|---|---|---|
| Samples | (n = 2) (Pa · s) | viscosity (Pa · s) |
| PEG-based Placebo | 5 150 | 5 110 |
| formulation | 5 070 | |
| RSD | — | 1.1% |
| PEG-based 10 mM | 10 394 | 10 219 |
| decitabine formulation | 10 045 | |
| RSD | — | 2.4% |

(RSD: relative standard deviation)

The complex viscosity of PEG-based 10 mM decitabine formulation is higher than its corresponding placebo. Even though the complex viscosity at rest is quite high, the oscillatory measurements showed that both samples under shear constraint are able to flow which is compatible with skin application. That means under rubbing, both samples are able to be easily spread onto the skin.

In summary, the complex viscosity measurements indicate that for topical application the complex viscosity should be in the range of from about 1,000 Pa·s to about 25,000 Pa·s at 25° C., preferably from about 2,000 Pa·s to about 15,000 Pa·s at 25° C., and even more preferably from about 3,000 Pa·s to about 11,000 Pa·s at 25° C. The complex viscosity may suitably be performed at room temperature (about 25° C.) under an oscillating ramp at a predefined strain amplitude fixed at 5 Pa and at a fixed frequency set at 1 Hz using a Rheometer (e.g. Anton Paar), in particular involving a cone-plate geometry with a gap 400 nm. The complex viscosity value is the mean of 10 values collected for 60 seconds.

### Example 3: Application of Decitabine Formulation According to the Invention on an Organotypic Culture (OTC) Model of HPV-Induced Pre-Cancerous Tumors

To confirm penetration of the drug substance Decitabine into the epithelial layer and induction of beneficial treatment effects on HPV-transformed cells upon topical application of a developed polyethylene glycol (PEG)-based Decitabine-containing formulation, organotypic culture (OTC) models were treated with different strengths of the drug product as detailed in Table 11 below. The employed OTC consist of keratinocytes and HPV-transformed SiHa cells co-cultivated on dermal equivalents (DE), thereby representing a three-dimensional (3D) in vitro model of an HPV-transformed pre-cancerous lesion that allows administration of topical formulations onto the air-exposed surface.

To enable topical application of the drug product onto the air-exposed surface of the OTC models in a standardized manner, small plates were generated from laboratory sealing film material to allow coverage of the whole air-exposed surface of the OTC. The plates were coated with the drug product, carefully placed on top of the OTC and removed after two hours of incubation (Table 11). The OTC models were treated topically with the PEG-based Decitabine-containing formulation at different strengths [0.1 mM, 1 mM or 10 mM] or a matching Placebo formulation as control over two weeks (5 applications/week, according to Table 11). The experiment was run in biological triplicates per condition. The OTC models were harvested on day 15 after the start of treatment and subsequently fixed in formalin and embedded in paraffin.

TABLE 11

| | | | Total | |
|---|---|---|---|---|
| Treatment | Route of | Treatment | Number of | Biological |
| Condition | Application | Duration | Applications | Replicates |
| Placebo | Topical | 2 weeks | 10 | 3 |
| 0.1 mM | Topical | 2 weeks | 10 | 3 |
| Decitabine | | | | |
| 1 mM | Topical | 2 weeks | 10 | 3 |
| Decitabine | | | | |
| 10 mM | Topical | 2 weeks | 10 | 3 |
| Decitabine | | | | |

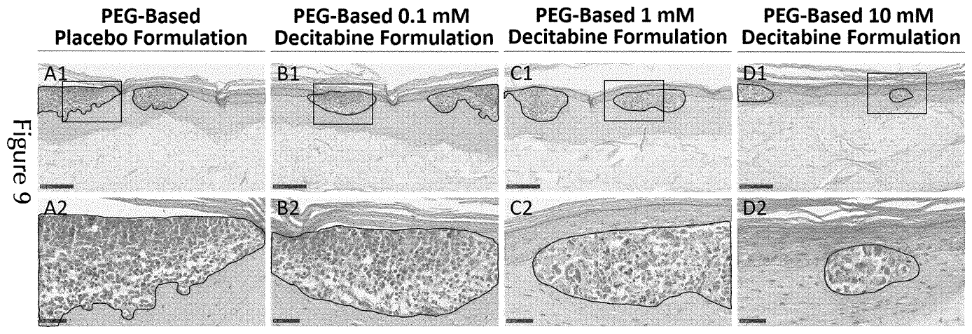

Tissue architecture and tumor cell morphology were assessed on sections from formalin-fixed and paraffin-embedded (FFPE) OTC that had been stained with hematoxylin and eosin (HE). Treatment effects of the PEG-based Decitabine-containing formulations on tissue architecture and tumor cell morphology in comparison to a matching Placebo formulation upon topical treatment are shown in FIG. 9. A dose-dependent degradation of tumor cells, as indicated by a loss of intact cell nuclei and accumulation of cell debris within the tumor nests (A->D) was observed. Notably, after the 2-week treatment period, epithelial stratification was still present in OTC models treated with the Placebo or different strengths of Decitabine and no detachment of the normal epithelium from the underlying dermal equivalent (DE) was observed in neither the Placebo nor the Decitabine-treated samples. In summary, the data show that treatment with a PEG-based, Decitabine-containing formulation according to the present invention leads to penetration of Decitabine into the epithelium and preferential degradation of HPV-transformed cells after topical application in an OTC model of HPV-transformed pre-cancerous tumors.

By the data set of Example 3, it is demonstrated that the inventive formulation is able to achieve the desired treatment effects on HPV-transformed cells (such as degradation of tumor cells) while epithelial integrity is maintained. The experiments are based on innovative organotypic culture (OTC) models mimicking HPV-induced pre-cancerous lesions morphologically. In this model, HPV-transformed cells are co-cultivated with normal human keratinocytes, thereby forming tumor cell nests that are surrounded by normally stratifying epithelium. This pre-cancerous model set-up allows the simultaneous assessment of treatment effects on HPV-transformed tumor cells and normal keratinocytes, and specifically enables the topical application of formulations. In a set of experiments with Decitabine-containing formulations, it was shown that sufficiently high dose of Decitabine is released from the inventive composition and delivered to the target cells to induce desired treatment effects in the HPV-transformed cells while relatively sparing surrounding normal epithelial cells. This discovery demonstrated veritable clinical potential of the invention for HPV-induced pre-cancerous lesions.

### Example 4 Measurement of Decitabine Stability and pH Under Controlled Storage Conditions

Stability of Decitabine (0.228 mg/g corresponding to 1 mM Decitabine) in a formulation as described by Table 2 (a polyethylene glycol-based formulation; ointment) was measured (at the start of the experiment (0), after one month (1) and after 3 months (3) at controlled storage conditions of (i) 5° C.+/−3° C. (Table 12) and (ii) of 25° C.+/−2° C. (60% RH+/−5%; RH: Relative Humidity) (Table 13). In addition to Decitabine stability, the pH of the formulation was determined at these time points. Decitabine stability was measured by HPLC as described by Example 1.

TABLE 12

Testing Timepoint (Months)

| | | |
|---|---|---|
| 0 | 1 | 3 |

| | |
|---|---|
| Tests | Results |

| | | | |
|---|---|---|---|
| pH | 7.1 | 7.1 | 7.0 |
| Decitabine assay (LC) [% LC] | 99.3 | 99.3 | 99.8 |

TABLE 13

Testing Timepoint (Months)

| | | |
|---|---|---|
| 0 | 1 | 3 |

| | |
|---|---|
| Tests | Results |

| | | | |
|---|---|---|---|
| pH | 7.1 | 7.1 | 7.0 |
| Decitabine assay (LC) [% LC] | 99.3 | 97.5 | 97.1 |

The stability of Decitabine is defined as “% LC”: Percent Label Claim defining the amount of Decitabine as the active ingredient provided in % within the tested formulation based on the initial amount of Decitabine as reference value (100%: 0.228 mg/g). The results clearly show that—irrespective of the storage conditions applied—an exceptionally high percentage of Decitabine is stably maintained over a period of up to 3 months. The pH of the formulation is essentially constant over the observation period. Thus, an unexpectedly high level of stability of the Decitabine formulation is achieved without the addition of any component other than PEG and DMSO.

### Example 5 Effects of Decitabine Formulation on Ex Vivo Cultivated HPV-Induced Pre-Cancerous Tumors

Example 5 studies the effects of a Decitabine formulation upon its topical administration on ex vivo cultivated HPV-induced pre-cancerous tumors.

### 1. Sample Preparation:

Biopsies obtained from patients with pre-cancerous lesions at the vulva (i.e. high-grade vulvar intraepithelial neoplasias, HG-VIN) were split into separate pieces using a scalpel. One part of the biopsies from each individual was fixed in formalin, embedded into paraffin.

The respective formalin-fixed paraffin-embedded (FFPE) tissue from this reference biopsy sample was tested for the presence of HPV DNA, genotyped for high-risk (HR) HPV types, and analyzed for the overexpression of the cellular protein p16^{INK4a} by immunohistochemistry. Combined positivity for HR-HPV DNA and p16^{INK4a} expression indicated HPV-induced transformation.

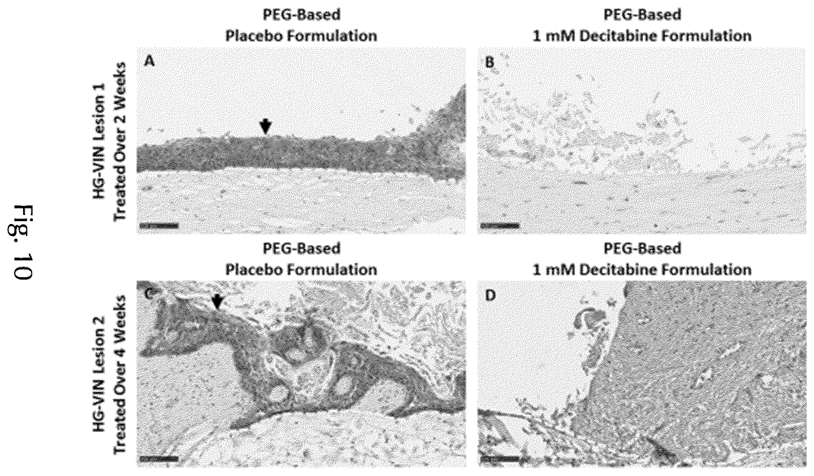

Further fresh (i.e. non-fixed) biopsy parts from the same individual were carefully placed on top of different dermal equivalents (DE; demarcated in the image from ex vivo cultivated lesion by dotted lining) and cultivated ex vivo for 3-5 days to allow for adhesion and outgrowth of lesional HPV-transformed epithelial cells over the surface of the DE. Subsequently, the respective cultures were treated topically ex vivo 5 times weekly over two (FIG. 10A, B) or four weeks (FIG. 10 C, D) with a PEG-based Decitabine-containing formulation as described by Table 2 (FIG. 10 B, D) at the strength of 1 mM or with a matching Placebo formulation as control (FIG. 10A, C)). Standardized topical administration was achieved by carefully placing formulation-loaded small plates generated from laboratory sealing film to the air-exposed surface of the ex vivo cultures allowing coverage of the whole epithelial surface with the formulation. After the end of the treatment period, the cultures were fixed in formalin and embedded in paraffin.

Tissue sections from the resulting FFPE blocks were stained for p16^{INK4a} by immunohistochemistry and subjected to whole-slide scanning. Tissue architecture and tumor cell morphology as well as expression of p16^{INK4a} protein were assessed on the scanned images and compared between the same samples from which biopsy parts were either treated with the PEG-based Decitabine-containing formulation [1 mM] (FIG. 10 B, D) or the matching Placebo formulation (FIG. 10A, C).

### 2. Results

FIG. 10 shows images of two HG-VIN biopsies cultivated and treated with the PEG-based Decitabine-containing formulation ([1 mM], FIG. 10 B and D) or the matching Placebo formulation (FIG. 10A and C) ex vivo over two or four weeks. Both lesions were demonstrated to be HPV-transformed (i.e. HR-HPV DNA-positive and p16^{INK4a}-positive) in the analyses on each corresponding reference biopsy sample. The HPV-transformed HG-VIN ex vivo cultures treated with the Placebo formulation over two (A) or four (C) weeks displayed a multi-layered epithelium with strong and diffuse nuclear and cytoplasmic expression of p16^{INK4a} (brown staining in original color images, visualized as dark grey staining in the presented black-and-white images and highlighted by arrows) indicative of vital HPV-transformed cells.

In contrast, ex vivo-cultivated HG-VIN biopsies from the same lesions treated with the PEG-based Decitabine-containing formulation [1 mM] over two (B) or four (D) weeks did not display any strong and diffuse nuclear and cytoplasmic expression patterns of p16^{INK4a}, Instead, evidence for tumor cell degradation, comprising the loss of intact cell nuclei and accumulation of cell debris on top of the DE was visible.

The obtained results indicate that topical treatment of ex vivo cultivated HPV-transformed pre-cancerous lesions with a PEG-based Decitabine-containing formulation according to the invention results in the penetration of Decitabine into the target cells, reversal of the HPV-transformed phenotype and corresponding, essentially complete degradation of tumor cells. These effects are shown by using the experimental approach of Example 5 as a genuine disease model based on patient samples and adopting the route of administration intended for the clinical setting (i.e. topical application).

1. Pharmaceutical composition comprising:
decitabine or azacitidine, or a pharmaceutically acceptable salt thereof;
polyethylene glycol (PEG); and
dimethyl sulfoxide (DMSO).

2. The pharmaceutical composition according to claim 1, wherein the composition comprises decitabine (or the salt thereof) at a concentration of 0.005-100 mM, preferably at a concentration of 0.01-50 mM, more preferably at a concentration of 0.1-10 mM (0.0228 mg/g-2.28 mg/g).

3. Pharmaceutical composition comprising 0.1-10 mM (0.0228 mg/g-2.28 mg/g) decitabine (or a pharmaceutically acceptable salt thereof) for medical use, wherein the pharmaceutical composition is applied topically.

4. The pharmaceutical composition according to anyone of the previous claims having a complex viscosity from about 1,000 Pa·s to about 25,000 Pa·s at 25° C., preferably from about 2,000 Pa·s to about 15,000 Pa·s at 25° C., and even more preferably from about 3,000 Pa·s to about 11,000 Pa·s at 25° C.

5. The pharmaceutical composition according to anyone of the previous claims comprising at least two distinct PEG components with a first PEG component having a solid physical state at 25° C. and a second PEG component having a liquid physical state at 25° C.

6. The pharmaceutical composition according to anyone of the previous claims comprising at least two distinct PEG components, wherein the first PEG component is PEG 1000-8000 and the second PEG component is PEG 200-600.

7. The pharmaceutical composition according to anyone of the previous claims comprising two distinct PEG components, wherein the first PEG component is PEG 1500 and the second PEG component is PEG 300.

8. The pharmaceutical composition according to anyone of the previous claims comprising DMSO at a concentration (weight by weight (w/w) of 1%-15%, preferably 2%-10%, more preferably 4%-7%, even more preferably 5%-6%, still more preferably about 5.7%.

9. The pharmaceutical composition according to anyone of the previous claims, wherein the composition is a non-liquid, semi-solid composition at 25° C.

10. The pharmaceutical composition according to anyone of the previous claims, wherein the composition does not comprise water.

11. The pharmaceutical composition according to anyone of the previous claims, wherein the composition does not comprise (any other) polar protic solvent, such as ethanol.

12. The pharmaceutical composition according to anyone of the previous claims, wherein the composition does not comprise any one of the following components, selected from the group consisting of a stabilizing agent other than PEG, an acidifying agent, a buffer salt, a pH modifier, a drying agent, an antioxidant, glycerin, and polypropylene glycol or a mixture thereof.

13. The pharmaceutical composition according to anyone of the previous claims, wherein the composition does not comprise any component other than decitabine or azacitidine (or the salt thereof), PEG and DMSO.

14. The pharmaceutical composition according to anyone of the previous claims comprising decitabine or azacitidine (or the salt thereof), PEG 1500, PEG 300 and DMSO.

15. The pharmaceutical composition according to anyone of the previous claims comprising or consisting of 0.1-10 mM (0.0228 mg/g-2.28 mg/g) decitabine (or azacitidine), 5.7% (w/w) DMSO, 40% (w/w) PEG 1500 and 54-55% (w/w) PEG 300.

16. The pharmaceutical composition of anyone of the previous claims for use in medicine.

17. The pharmaceutical composition of anyone of claims 1 to 15 for use according to claim 16, wherein the pharmaceutical composition is applied topically.

18. The pharmaceutical composition of anyone of claims 1 to 15 for use according to claim 16 or 17, wherein the composition is for use in the treatment of a medical condition or disorder of the keratinizing and non-keratinizing epithelium/skin.

19. The pharmaceutical composition of anyone of the previous claims for use in the treatment of human papillomavirus (HPV)-related pre-cancerous conditions, wherein the pharmaceutical composition is applied topically.

20. The pharmaceutical composition of anyone of claims 1 to 15 for use according to anyone of claims 16 to 19, wherein the composition is applied on the anogenital area, in particular on the vulva.

21. A method for preparing the pharmaceutical composition according to anyone of the previous claims, the method comprising the following steps:
(1) preparing a solution of decitabine or azacitidine (or the salt thereof) in DMSO;
(2) melting the PEG component(s) to obtain a homogeneous composition; and
(3) mixing the decitabine (or azacitidine) solution of step (1) with the melted PEG component(s) of step (2) to obtain a homogenous composition.

22. A package containing the pharmaceutical composition according to anyone of claims 1 to 15.

23. The package according to claim 22, wherein the package is designed as a deformable tube.
